(19) United States
(12) Patent Application Publication
(10) Pub. No.: US 2002/0012911 A1
(43) Pub. Date: Jan. 31, 2002

(54) NOVEL METHOD FOR THE PRESELECTION OF SHOTGUN CLONES OF THE GENOME OR A PORTION THEREOF OF AN ORGANISM

(76) Inventors: UWE RADELOF, KLEINMACHNOW (DE); HANS LEHRACH, BERLIN (DE); STEFFEN HENNIG, BERLIN (DE); MATTHIAS STEINFATH, BERLIN (DE); FIONA FRANCIS, PARIS (FR); ANNEMARIE POUSTKA, HEIDELBERG (DE); PETER SERANSKI, HEIDELBERG (DE); DOLORES CAHILL, BERLIN (DE)

Correspondence Address: FISH & RICHARDSON, PC, 4350 LA JOLLA VILLAGE DRIVE, SUITE 500, SAN DIEGO, CA, 92122, US

(21) Appl. No.: 09/277,689

(22) Filed: Mar. 26, 1999

(30) Foreign Application Priority Data
Sep. 29, 1998 (US) PCT/EP98/06146

(51) Int. Cl. C12Q 1/68

(52) U.S. Cl. 435/6

(57) ABSTRACT

The present invention relates to a method for the preselection of shotgun clones, e.g., cosmids, PACs, BACs, etc. of a genome of an organism, or of parts of the genome of an organism that significantly reduces the time and workload associated with the further processing of shotgun clones, for example, in sequencing projects such as the human genome project. The invention relies on a combination of steps including the transfer of shotgun clones to a carrier, e.g., nylon membrane, glass chip, etc. where the clones bind, preferably hybridize to a set of specifically selected probes, e.g., DNA oligonucleotides, PNA oligonucleotides or pools of DNA or/and PNA oligonucleotides, further antibodies, fragments or derivatives thereof which are labeled or unlabeled. Each probe of said set interacts to 1 to 99% (ideally 50%) of all shotgun clones (nucleic acid fragments) in all investigated shotgun libraries, Clones that are characterized as being divergent as a result of the binding experiment in all likelihood represent different parts of the genome or of the investigated part of the genome. The preselection for such divergent clones will reduce the number of redundant analysis of, e.g., DNA sequences.

This specification cites a number of published references. All these references are Incorporated herein by reference.

The present invention relates to a method for the preselection of shotgun clones, e.g., cosmids, PACs, BACs, etc. of a genome of an organism, or of parts of the genome of an organism that significantly reduces the time and workload associated with the further processing of shotgun clones, for example, In sequencing projects such as the human genome project. The invention relies on a combination of steps including the transfer of shotgun clones to a carrier, e.g., nylon membrane, glass chip, etc. where the clones bind, preferably hybridize to a set of specifically selected probes, e.g., DNA oligonucleotides, PNA oligonucleotides or pools of DNA or/and PNA oligonucleotides, further antibodies, fragments or derivatives thereof which are labeled or unlabeled. Each probe of said set interacts to 1 to 99% (ideally 50%) of all shotgun clones (nucleic acid fragments) in all investigated shotgun libraries. Clones that are characterized as being divergent as a result of the binding experiment in all likelihood represent different parts of the genome or of the investigated part of the genome. The preselection for such divergent clones will reduce the number of redundant analysis of, e.g., DNA sequences.

Since the foundation of the Human Genome Organisation (HUGO) in McKsuick V. A., Genomics 5(2) (1989), 385 less then 5 percent of the human genome has been sequenced (Beck S., http:l/www.ebl.ac.uk/-sterklgenome-MOT/ (1998)). Completion of the project until 2005 will therefore require either appropriate increases in funding or the use of new methods (3,4).

In spite of a number of alternative proposals for directed sequencing strategies like deterministic sequencing (Frischauf A.M. et al., Nucleic Acids Res. 8(23) (1980), 5541), transposon-facilitated sequencing (Phadnis S. H. et al., Proc, Natl. Acad. Sci. USA 86(15) (1989), 5908; Kleckner N. et al., Methods Enzymol. 204 (1991) 139; Strathmann M. et al., Proc. Natl. Acad. Sci. USA 88(4) (1991), 1247; Devine S. E. et al., Nucleic Acids Res. 22(18) (1994), 3765), primer walking and primer ligation (Bloecker H. et al., Computer Applications in the Biosciences 10(2) (1994), 1939). most sequence information has been generated by traditional shotgun sequencing. As an inherent part of this method longer sequences have to be subdivided into shorter, overlapping sequence stretches. If that subdivision is random, as in the case of traditional shotgun sequencing, an unequal representation of different parts of the sequence will be expected due to sampling effects, requiring oversampling to ensure a minimal coverage of underrepresented regions. This situation can be considerably worse because of biological effects, e.g., different cloning efficiencies of different sequence stretches. Typically more than 2000 sequence reads per 100 kb are generated from randomly chosen shotgun clones and assembled in order to reconstruct the entire genomic sequence. To close the remaining gaps in the consensus sequence directed approaches are used such as primer walking. Completed shotgun projects show an 8-12 fold average coverage per base final sequence which is significantly more redundant than necessary to achieve consensus sequence data of sufficient quality. In addition, it is a common situation in large-scale sequencing projects that the target region be spanned by overlapping genomic clones (cosmids, PACs, etc.), and it is often difficult to find a set of those clones which cover long sequence stretches with a minimal amount of overlap. The resulting redundancy in the overlapping regions is twice as high as in the nonoverlapping regions.

As a very useful advance, a subset of shotgun clones with no or little overlap can be selected from shotgun libraries, using automated facilities (Lehrach H. et al., Genome Analysis, Cold Spring Harbor Laboratory Press, Cold Spring Harbor 1 (1990), 39) to generate and analyze high density filter arrays.

A sampling without replacement method was introduced by (Hoheisel J. D. et al., Cell 73(1) (1993). 109) and applied to shotgun clone selection by (Scholler P. et al., Nucleic Acids Res. 23(19) (1995), 3842). In this strategy individual clones or pools of clones of fixed length are used as hybridization probes. The number of experiments (clone-probe tests), is therefore proportional to N², the square of the number of clones analyzed in each individual shotgun library. If clone pools are used as hybridization probes, the effort is reduced by a constant factor. The approach requires the generation of new probes for each new library, and requires therefore a quite significant upstream effort. Moreover, it will often have difficulties with repeat sequences in the probes and the procedure works sequentially. The result of one hybridization experiment has to be analyzed before the next one can be carried out.

In summary, a variety of methods have been established in the art to diminish the problems and workload associated with sequencing of such DNA molecules. However, the methods developed so far lack efficiency (they are complicated, and require significant efforts and costs). Alternatively, they were generally not believed applicable to the sequencing of genomic DNA without further sophistication. Accordingly, the costs associated with these processes is still considerably high.

Therefore, the technical problem underlying the present invention was to establish a simple method for reducing efforts and the costs associated with the sequencing of large genomic structures. The solution to this technical problem is achieved by providing the embodiments characterized in the claims.

Accordingly, the present invention relates to a method for the preselection of shotgun clones of the genome or a portion of a genome of an organism comprising:

(a) providing a shotgun library of said genome or said portion of the genome:

(b) amplifying said library by an amplification method;

(c) transferring clones of said library onto a carrier;

(d) optionally, generating one or more replicas of said carrier;

(e) allowing binding of a set of labeled or unlabeled probes

(ea) sequentially to said clones on said carrier or clones on replica(s) of said carrier(s); or/and

(eb) to clones on said carrier and to clones on replicas of said carrier or to clones on replicas of said carrier;

(f) detecting clones that bind to one or more of said probes,

(g) optionally, evaluating the signal intensity of said binding;

(h) selecting a number of clones that were detected in step (f), or evaluated in step (g) wherein

(ha) each of said clones binds with at least one different probe of said set of probes; or

(hb) clones that bind to the same probes from said set of probes generate different signal intensities in the binding signal with at least one probe from said set of probes; and

wherein the sum of the basepairs of the inserts of said shotgun clones at least equals the number of basepairs of the genome or the portion of the genome of said organism.

The amplification may be a DNA amplification or may be an amplification of hosts carrying the DNA.

The carrier is referred to above is usually a solid carrier,

The term “portion of a genome” as used herein denotes a portion that is at least 1 kb. Preferably, such a portion is a part of or a complete eukaryotic chromosome.

The term “shotgun library” is understood by the person skilled in the art to denote a shotgun library from a variety of sources such as eukaryotic genomes or parts thereof.

The term “DNA amplification method” relates to any known method of amplifying DNA such as ligase chain reaction or polymerase chain reaction (PCR). Although it is desirable that all clones/DNAs are amplified at equal frequency, it is known that this is not (always) the case. Accordingly, the term “amplifying said library” also relates to embodiments were not all members of said library are amplified or are not amplified at equal frequency.

The term “clone” refers to nucleic acid molecules, preferably DNA as well as to hosts comprising such nucleic acid molecules such as bacteria, preferably E. coli, viruses, phage or eukaryotic cells such as yeast cells, fungal cells, mammalian cells or insect cells and thus, for example, to transformed or transfected cells.

The term “generating one or more replicas of said carrier” means in accordance with the present invention that said carrier replica (e.g., another filter) comprises clones attached thereto in the same array as on the carrier that is mentioned in step (c).

The difference in steps (ea) and (eb) arises from the fact, that in the first case, different probes are allowed to bind to the same carrier or to the same replicas of said carrier sequentially. In other words, after the binding and detection of a signal, the probe is removed from the carrier and the DNA on the carrier allowed to bind with another labeled or unlabeled probe which subsequently is detected according to known methods or methods described herein. The location of the signal-generating clone should be retained, e.g., by autoradiography, prior to removal of the probe. Removal of probes is well known in the art and described, for example, in Sambrook et al., “Molecular Cloning, A Laboratory Handbook”, 2^{nd} ed. 1989, CSH Press, Cold Spring Harbor, N.Y. Conveniently, filters are allowed to bind with more than one probe, preferably up to five different probes. If option (eb) is employed, i.e. if each carrier is used only once for binding, then a sufficient amount of carriers has to be employed that allows a number of binding reactions permitting a meaningful preselection of clones. The amount of selected clones is preferably in the range from 384 to 600 clones depending on the size of the library. The present invention also envisages combinations of (ea) and (eb).

A difference in the signal intensity allows conclusions with respect to the complementarity of probe and sample. For example, a mismatch may lead to a less efficient hybridization which is one example of the binding reaction and therefore to a weaker signal than a hybridization without mismatch. A difference in the signal intensity may therefore be interpreted as a difference in the DNA sequence of the samples. Both samples may consequently be further investigated

The method of the present invention is a powerful combination of oligonucleotide fingerprinting and shotgun sequencing. To select optimal sets of shotgun clones prior to sequencing, the prior art teaches that clones from shotgun libraries could be ordered into contigs, based on the results of an oligofingerprinting experiment (Poustka A. et al., Cold Spring Harb, Symp. Quant Biol. 51(Pt1) (1986), 131). This however, requires an unacceptably large number of hybridization experiments, and would partly generate information on exact overlaps between clones, which is then independently generated again in the sequencing procedure. This unacceptably large number is reduced to an acceptable number by employing the method of the present invention. Although a variety of methods for large scale sequencing were available in the art, none of these methods proved to be as cost efficient and, at the same time, easy to use as the method of the present invention. Alternatively, methods employed for sequencing cDNA libraries were deemed not applicable to whole genome or portions of genomes due to the much higher complexity of the genomic structures as compared, for example, to cDNA.

Sequence information generated and oligofingerprinting results can now be combined to select clones in regions of weak quality sequence-data and for bridging or extending into gap regions. The method of the invention can therefore aid in gap closure.

Even with the simple analysis software used in the actual experiments underlying the present invention, the approach of the invention “preselection by oligonucleotide fingerprinting” (PrOF) has resulted in significant cost reductions and throughput improvements in large-scale sequencing. It was demonstrated both in simulations and large scale experiments that the number of clones to be sequenced in shotgun projects can be significantly reduced. The reduction can be increased further if genomic regions spanned by overlapping genomic clones are being sequenced, because shotgun clones are distinguished solely by their oligofingerprint and selected with the same average redundancy in the overlap region of two libraries as for the nonoverlapping regions.

The nucleic acid molecules, preferably comprised in the host cell are preferably affixed to a planar carrier. As is well known in the art, said planar carrier to which said nucleic acid may be affixed, can be for example, a Nylon-, nitrocellusose- or PVDF membrane, glass or silica substrates (DeRisl et al., Nat. Genet. 14 (1996), 457-460; Lockhart et al., Nature Biotechnology 12 (1996), 1675-1680). Said host cells containing said nucleic acid may be transferred to said planar carrier and subsequently lysed on the carrier and the nucleic acid released by said lysis is affixed to the same position by appropriate treatment. Alternatively, progeny of the host cells may be lysed in a storage compartment and the crude or purified nucleic acid obtained is then transferred and subsequently affixed to said planar carrier. Advantageously, said nucleic acids are amplified by PCR prior to transfer to the planar carrier. As is well known in the art, such regular grid patterns may be at densities of between 1 and 50,000 elements per square centimeter and can be made by a variety of methods. Preferably, said regular patterns are constructed using automation or a spotting robot such as described in Lehrach et al., Science Rev. 22 (1997), 37-43 and Maier et al., Drug Disc. Today 2 (1997), 315-324 and furnished with defined spotting patterns, barcode reading and data recording abilities. Thus it is possible to correctly and unambiguously return to stored host cells containing said nucleic acid from a given spotted position on the planar carrier. Also preferably, said regular grid patterns may be made by pipetting systems, or by microarraying technologies as described by Shalon et al., Genome Research 6 (1996), 639-645, Schober et al., Biotechniques 15 (1993), 324-329 or Lockart et al., Nature Biotechnology 12 (1996), 1675-1680.

The method has proved to be more efficient than a sampling without replacement strategy due to a more favorable scaling behavior (NlogN instead of N²), the use of a standard set of probes for all experiments and, as shown in the appended examples, a reduced sensitivity to the effect of repeat rich genomic regions, shotgun clone insert sizes and insert size distributions.

A main advantage of the method of the invention is the rapid handling of many shotgun libraries in massively parallel experiments. Moreover, once the technical facilities required are available in a sequencing laboratory the preselection costs, Including all materials and salaries, are about 5% of the cost of traditional shotgun sequencing if one carrier, preferably a filter (capacity about 900 kb) is handled as in the experiments described here. The costs per filter are much further reduced if multiple filters are handled in parallel. For example, 4 different filters may routinely be hybridized in one hybridization bottle, using the same amount of chemicals used here for one filter. It is feasible for the skilled person to perform the oligofingerprinting of batches of shotgun libraries representing a total sequence length of more than 3.5 Mb in parallel within two months including all working steps from the amplification, preferably PCR to the re-arraying of the selected clones. This additional effort and cost at least doubles the sequencing throughput independently from the sequencing technology used, because less than half the number of clones have to be sequenced now. The technique is also expected to be useful in very large-scale sequencing projects, as for example in whole genome shotgun sequencing projects proposed for the human genome by Weber et al., Genome Res. 7(5) (1997), 401-9 and planned now by Venter et al., Science 280(5369) (1998), 1540-2 after criticism by (Green, Genome Res. 7(5) (1997), 410). To be able to approach such large projects, further Improvements in the software, but also in the throughput of the oligofingerprinting pre-screening (clone picking, PCR, spotting, hybridization, e.g,, use of fluorescent labeled oligonucleotides and fully automated hybridization) will still be helpful, although not required for the present invention.

Whereas some of the embodiments of the present invention described above specifically refer to nucleic acid hybridization wherein the probe is a nucleic acid such as an oligonucleotide which advantageously is labeled, the probe may also be any of the other recited molecule types. Depending on the type of molecules employed, the conditions which allow binding of said probe to said clone/DNA will vary. For example, if an antibody is used as a probe, the binding conditions will be different than those used in nucleic acid hybridization. Antibodies or fragments or derivatives thereof such as Fab, F(ab)₂ or Fv fragment or scFv fragments may be used to detect, for example, DNAs forming zinc finger motifs. Stronger or weaker signals obtained with antibodies may be due to the fact that an antibody binds strongly or less strongly to a certain epitope generated by the DNA. Cross-reactions of antibodies may also result in different signal intensities. As regards the teachings of the present invention with respect to the application of antibodies as probes, it is referred to Harlow and Lane “Antibodies, A Laboratory Manual”, CHS Press, Cold Spring Harbor, N.Y., 1988.

The probes may be labeled or unlabeled. Labeling of nucleic acids or antibodies is very well known in the art and described in Sambrook, loc. cit. or Harlow and Lane, loc. cit. Commonly used labels comprise, inter alia, fluorochromes (like fluorescein, rhodamine, Texas Red, etc.) enzymes (like horse radish peroxidase, β-galactosidase, alkaline phosphatase), radioactive isotopes (like ³²P or ¹²⁵I), biotin, digoxygenin, colloidal metals, chemi- or bioluminescent compounds (like dioxetanes, luminol or acridiniums). Labeling procedures, like covalent coupling of enzymes or biotinyl groups, lodinations, phosphorylations, biotinylations, random priming, nick-translations, tailing (using terminal transferases) are well known in the art.

Detection methods comprise, but are not limited to, autoradiography, fluorescence microscopy, direct and indirect enzymatic reactions, etc.

If the probes are unlabeled, then a system must be provided such that the probes or the interaction of the probes with the DNA molecules provide the signal. An example of the provision of such a signal is by means of mass spectrometry (Mass Spectometry, Duckworth, Barber and Venkatasubramanian, Cambridge Monographs on physics, 2^{nd} ed., 1990).

The term “hybridizing” preferably relates to stringent or nonstringent hybridization conditions. Examples of such conditions are known to the person skilled in the art. The person skilled in the art may devise such conditions on the basis of his common general knowledge including textbooks such as Sambrook et al., “Molecular Cloning, A Laboratory Handbook”, 2^{nd} ad. 1989, CSH Press, Cold Spring Harbor, N.Y. or Hames and Higgins (ads.). “Nucleic acid hybridization, a practical approach”, IRL Press, Oxford, Washington, D.C., 1985. The setting of conditions is well within the skill of the artisan and to be determined according to protocols described in the art. Thus, the detection of only specifically hybridizing sequences will usually require stringent hybridization and washing conditions such as 0.1× SSC, 0.1% SDS at 65°. Non-stringent hybridization conditions for the detection of homologous or not exactly complementary sequences may be set at 6× SSC, 1% SDS at 65° C. As is well known, the length of the probe and the composition of the nucleic acid to be determined constitute further parameters of the hybridization conditions.

In a preferred embodiment of the method of the present invention said organism is a mammal, preferably a human or mouse, a zebrafish, drosophila, amphioxus, a plant, preferably arabidopsis, a fungus, preferably yeast, or a microorganism, preferably a bacterium, preferably meningococcus.

In a further preferred embodiment said shotgun library is provided in a storage compartment.

The host cells carrying the shotgun library will, in this preferred embodiment, be propagated in said storage compartment and provide further progeny for additional tests. Of course, the further steps of the method of the invention may be carried out immediately after transfer of the clones into the storage compartment. Preferably, replicas of said storage compartment maintaining the array of clones are set up, Said storage compartments comprising the transformed host cells and the appropriate media may be maintained in accordance with conventional cultivation protocols. Alternatively, said storage compartments may comprise an anti-freeze agent and therefore be appropriate for storage in a deep-freezer. This embodiment is particularly useful when the evaluation of the DNA sequences is to be postponed. As is well known in the art, frozen host cells may easily be recovered upon thawing and further tested in accordance with the invention. Most preferably, said antifreeze agent is glycerol which is preferably present in said media in an amount of 3-25% (vol/vol).

In a particularly preferred embodiment said storage department is the microtiter plate. Most preferably, said microtiter plate comprises 384 wells. Microtiter plates have the particular advantage of providing a pre-fixed array that allows the easy replicating of clones and furthermore the unambiguous identification and assignment of clones throughout the various steps of the experiment. The 384 well microtiter plate is, due to its comparatively small size and large number of compartments, particularly suitable for experiments where large numbers of clones need to be screened.

Depending on the design of the experiment, the host cells may be grown in the storage compartment such as the above microtiter plate to logarithmic or stationary phase. Growth conditions may be established by the person skilled in the art according to conventional procedures. Cell growth is usually performed between 15 and 45° C.

Whereas the optionally labeled oligonucleotides may be of varying length and conveniently may comprise up to 25 nucleotides, in another preferred embodiment said oligonucleotides comprise between 2 and 50 nucleotides. More preferably, said oligonucleotides comprise between 6 and 10 nucleotides.

In an additional preferred embodiment of the invention, said carrier is a planar carrier.

It is particularly preferred that said planar carrier is a nylon membrane, or filter, or chip, or beads, or glass, or silicon, or metal, or plastic or ceramics, or specially treated or coated versions of the aforementioned.

In an additional particularly preferred embodiment said filter is a nylon filter or a nylon membrane.

Another preferred embodiment is that said transfer in step (c) is made or assisted by automation, spotting robot, pipetting or micropipetting device. How such a spotting robot may be devised and equipped is, for example, described in Lehrach et al., Science Rev. 22 (1997), 37. Naturally, other automation or robotic systems that reliably create ordered arrays of clones may also be employed.

In a further preferred embodiment said transfer is in a regular grip pattern.

Most advantageously, said transfer is effected in a regular grid pattern at densities of 1 to 1,000,000, preferably 10 to 10,000 spots of PCR products (or otherwise generated nucleic acid fragments) of shotgun clones per square centimeter. The progeny of said host cells may be transferred to a variety of (planar) carriers. Most preferred is a membrane which may, for example, be manufactured from nylon, nitro-cellulose or PVDF.

The way the probes (oligonucleotides) are selected is based on the following idea: The highest information value of a single hybridization experiment could be achieved using an oligonucleotide (or even a pool of different oligonucleotides) that has a hybridization probability of 50% to all clones in the shotgun libraries in question. Therefore, this probe divides all clones in 2 partitions of the same size (clones with/without a hybridization signal). The ideal set would consist of probes each having that hybridization probability. In addition, every single probe would, together with a second one, divide all clones in four partitions of the same size and together with a third one in 8 partitions of the same size etc. On the basis of this teaching and using this general knowledge, the person skilled in the art is in the position to devise appropriate oligonucleotide probes. An example how such a selection may be effected is provided herein below.

Referring now to the step (f) of the method of the invention, the readout system for detecting the clones, namely the label attached to the probes can be analyzed by a variety of means. For example, it can be analyzed by visual imaging or inspection, radioactive, chemituminescent, bioluminescent, fluorescent, photometric, spectrometric, infra red, colourimetric or resonant detection. In a preferred embodiment said probes are unlabeled or labeled with a radioactive, a chemiluminescent, a bioluminescent, a fluorescent, a phosphorescent marker or a mass label.

In a further preferred embodiment said detection is effected by digital image storage, analysis, processing or mass spectrometry.

In an additional preferred embodiment said set of probes comprises between 10 and 10,000 different probes such as 15, 20, 50, 100, 1000 or 5000 different probes.

In a further preferred embodiment, In step (d) between 1 and 10,000 replicas are generated. In another preferred embodiment, in step (d) between 2 and 10,000 different replicas are generated such as 3, 4, 5, 6, 7, 8. 9, 10, 20, 100 or 1000 replicas.

In another preferred embodiment the sum of basepairs of said inserts amounts to 1 to 30 times the number of basepairs in the genome or said portion of the genome of said organism.

In a particularly preferred embodiment the sum of basepairs of said inserts amounts to 2 to 4 times the number of basepairs in the genome or said portion of said genome of said organism.

The term “insert” is used as in conventional molecular biology and denotes a nucleic acid molecule of potential interest that is contained in a vector. Here, the inserts are derived from the genome or the portion of said genome.

In a preferred embodiment said amplification, preferably DNA amplification, in step (b) is effected by polymerase chain reaction (PCR).

Another preferred embodiment of the invention relates to a method further comprising

(i) sequencing clones selected after hybridizing to said oligonucleotides/probes. Sequencing of DNA is well known in the art and described, e.g., In Sambrook, loc. cit. Advantageously, the complete genome or the complete portion of the genome from which the shotgun library is derived is sequenced by this method.

In a particularly preferred embodiment said probe, preferably said oligonucleotide recognizes a contiguous or non-contiguous region of between 2 and 30 nucleotides,

In another particularly preferred embodiment each clone binds to a different subset of probes indicating minimal overlap to previously selected clones based on appropriate statistical criteria to produce a minimal overlapping clone set.

Further, the invention relates to a method for the production of a composition, preferably a pharmaceutical composition comprising formulating an open-reading frame (ORF) comprised in a clone selected after hybridizing to one of said oligonucleotides or an expression product thereof in a pharmaceutically acceptable form.

The components of the composition of the invention may be packaged in containers such as vials, optionally in buffers and/or solutions. If appropriate, one or more of said components may be packaged in one and the same container.

Optionally, the ORF is cloned in an (expression) vector. Vectors, particularly plasmids, cosmids, viruses and bacteriophages are used conventionally in genetic engineering. Preferably, said vector is an expression vector and/or a gene transfer or targeting vector. Expression vectors derived from viruses such as retroviruses, vaccinia virus, adeno-associated virus, herpes viruses, or bovine papilloma virus, may be used for delivery of the polynucleotides or vector of the invention into targeted cell population. Methods which are well known to those skilled in the art can be used to construct recombinant viral vectors; see, for example, the techniques described in Sambrook et al., Molecular Cloning A Laboratory Manual, Cold Spring Harbor Laboratory (1989) N.Y, and Ausubel et al., Current Protocols in Molecular Biology, Green Publishing Associates and Wiley interscience, N.Y. (1989). Alternatively, the polynucleotides and vectors of the invention can be reconstituted into liposomes for delivery to target cells. The vectors containing the polynucleotides of the invention can be transferred into the host cell by well-known methods, which vary depending on the type of cellular host. For example, calcium chloride transfection is commonly utilized for prokaryotic cells, whereas, e.g., calcium phosphate or DEAE-Dextran mediated transfection or electroporation may be used for other cellular hosts; see Sambrook, supra.

Such vectors may comprise further genes such as marker genes which allow for the selection of said vector in a suitable host cell and under suitable conditions. Preferably, the polynucleotide to be preselected is operatively linked to expression control sequences allowing expression in prokaryotic or eukaryotic cells. Expression of said polynucleotide comprises transcription of the polynucleotide into a translatable mRNA. Regulatory elements ensuring expression in eukaryotic cells, preferably mammalian cells, are well known to those skilled in the art. They usually comprise regulatory sequences ensuring initiation of transcription and, optionally, a poly-A signal ensuring termination of transcription and stabilization of the transcript, and/or an intron further enhancing expression of said polynucleotide. Additional regulatory elements may include transcriptional as well as translational enhancers, and/or naturally-associated or haterologous promoter regions. Possible regulatory elements permitting expression in prokaryotic host cells comprise, e.g. the PL, lac, trp or tac promoter in E. coli, and examples for regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. Beside elements which are responsible for the initiation of transcription such regulatory elements may also comprise transcription termination signals, such as the SV40-poly-A site or the tk-poly-A site, downstream of the polynucleotide. Furthermore, depending on the expression system used leader sequences capable of directing the polypeptide to a cellular compartment or secreting it into the medium may be added to the coding sequence of the polynucleotide of the invention and are well known in the art. The leader sequence(s) is (are) assembled in appropriate phase with translation, initiation and termination sequences, and preferably, a leader sequence capable of directing secretion of translated protein, or a portion thereof, into the periplasmic space or extracellular medium. Optionally, the heterologous sequence can encode a fusion protein including an C- or N-terminal identification peptide imparting desired characteristics, e.g., stabilization or simplified purification of expressed recombinant product. In this context, suitable expression vectors are known in the art such as Okayama-Berg cDNA expression vector pcDV1 (Pharmacia), pCDM8, pRc/CMV, pcDNA1, pcDNA3 (In-vitrogene), pSPORT1 (GIBCO BRL)) or pCl (Promega).

Preferably, the expression control sequences will be eukaryotic promoter systems in vectors capable of transforming or transfecting eukaryotic host cells, but control sequences for prokaryotic hosts may also be used.

As mentioned above, the vector of the present invention may also be a gene transfer or targeting vector. Gene therapy, which is based on introducing therapeutic genes into cells by ex-vivo or in-vivo techniques is one of the most important applications of gene transfer Suitable vectors and methods for in-vitro or in-vivo gene therapy are described in the literature and are known to the person skilled in the art; see, e.g., Giordano, Nature Medicine 2 (1996), 534-539; Schaper, Circ. Res. 79 (1996), 911-919; Anderson, Science 256 (1992), 808-813; Isner, Lancet 348 (1996), 370-374; Muhihauser, Circ. Res. 77 (1995), 1077-1086; Wang, Nature Medicine 2 (1996), 714-716; WO94/29469: WO 97/00957 or Schaper, Current Opinion in Biotechnology 7 (1996), 635-640, and references cited therein. The polynucleotides and vectors of the Invention may be designed for direct introduction or for introduction via liposomes, or viral vectors (e.g., adenoviral, retroviral) into the cell. Preferably, said cell is a germ line cell, embryonic cell, or egg cell or derived therefrom, most preferably said cell is a stem cell.

The pharmaceutical composition of the present invention may further comprise a pharmaceutically acceptable carrier and/or diluent. Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous. intraperitoneal, subcutaneous, intramuscular, topical, intradermal, intranasal or intrabronchial administration. The dosage regimen will be determined by the attending physician and clinical factors, As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 0.001 to 1000 μg (or of nucleic acid for expression or for inhibition of expression in this range); however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Progress can be monitored by periodic assessment. Dosages will vary but a preferred dosage for intravenous administration of DNA is from approximately 10⁸ to 10¹² copies of the DNA molecule. The compositions of the invention may be administered locally or systemically. Administration will generally be parenterally, e.g., intravenously; DNA may also be administered directly to the target site, e.g., by biolistic delivery to an internal or external target site or by catheter to a site in an artery. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, Including saline and buffered media. Parenteral vehicles include sodium chloride solutions Ringer's dextrose, dextrose and sodium chloride, lactated Ringer's, or fixed oils. Intravenous vehicles Include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents such as interleukins or interferons depending on the intended use of the pharmaceutical composition,

The figures show:

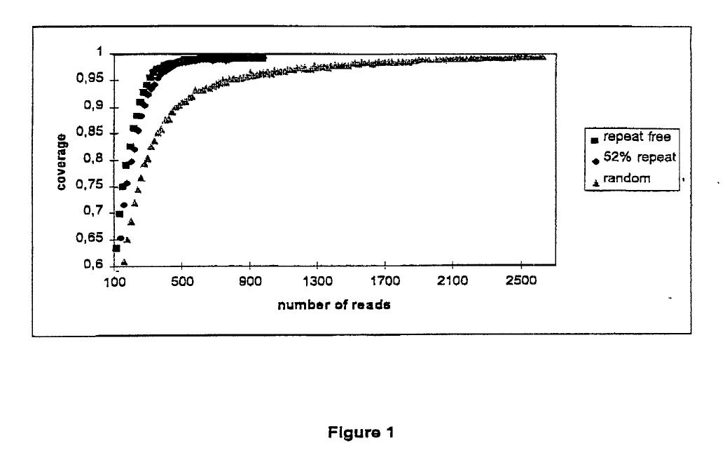

FIG. 1 Influence of repeat content on preselection efficiency: A 100 kb genomic sequence with a repeat content of 52% was used in comparison to a 100 kb artificially repeat free sequence. The number of reads (x-axis) necessary to achieve a certain percentage of the whole sequence (y-axis) is plotted. Each point of the curves represents the average value of 50 statistically independent experiments. The efficiency of random selection used in the standard shotgun approach is also shown.

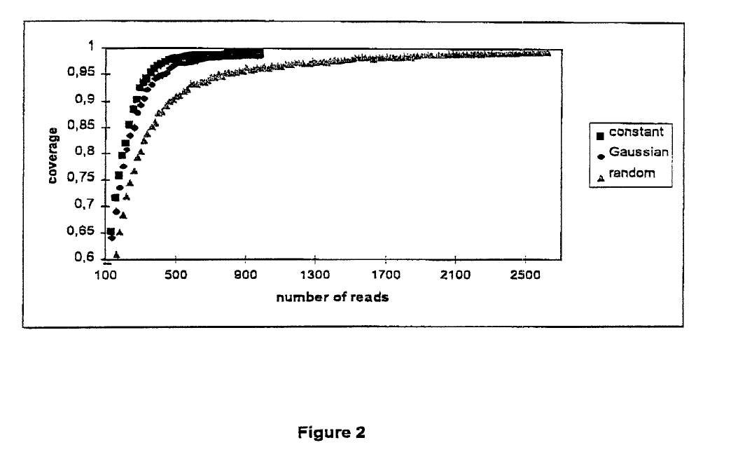

FIG. 2 Influence of clone length distribution on selection efficiency: The same 100 kb genomic sequence of 52% repeats used in FIG. 1 was cut into shotgun clones of fixed insert length of 1.5 kb in case 1 and into clones of Gaussian distributed insert length centered around 1.5 kb (σ=200 bp) In case 2. The number of reads (x-axis) necessary to achieve a certain percentage of the whole sequence (y-axis) is plotted. Each point of the curves represents the average value of 50 statistically independent experiments. The efficiency of random selection used in the standard shotgun approach is also shown. In this case a fixed insert length of 1.5 kb is used.

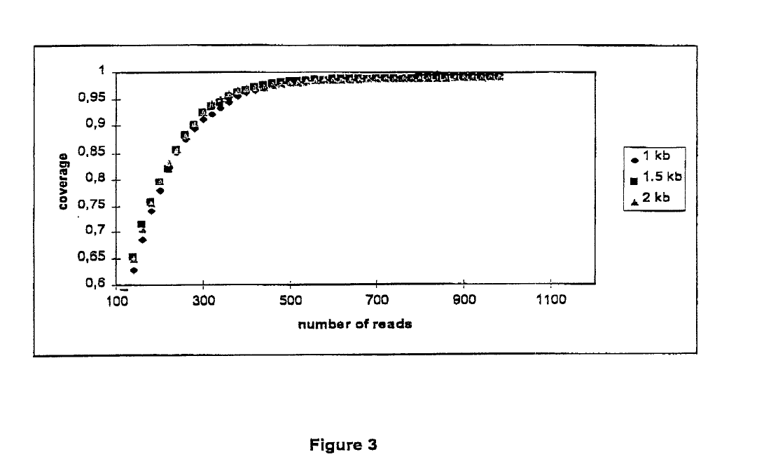

FIG. 3 Influence of shotgun clone insert size: The same 100 kb genomic sequence of 52% repeats used in FIGS. 1 and 2 was cut into shotgun clones of different (1 kb, 1.5 kb and 2 kb) but fixed sizes. The number of reads (x-axis) necessary to achieve a certain percentage of the whole sequence (y-axis) is plotted. Each point of the curves represent the average value of 50 statistically independent experiments.

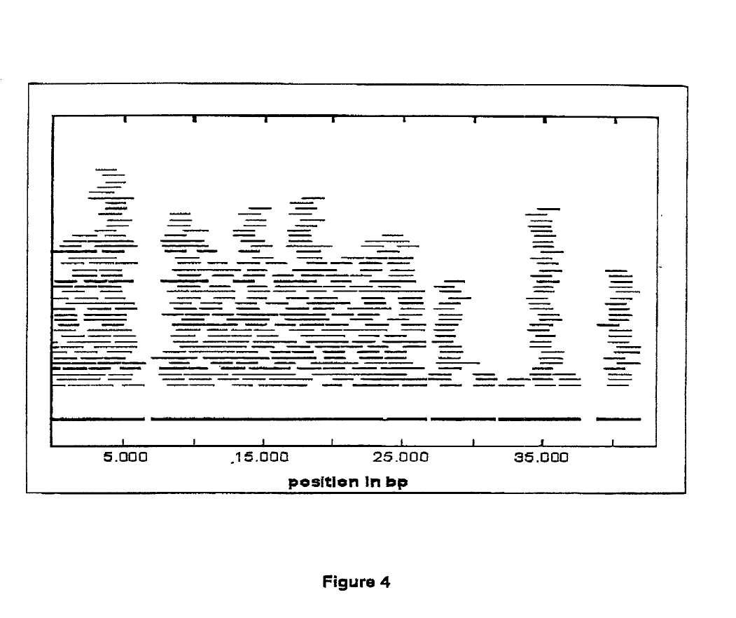

FIG. 4 Assembly of 426 shotgun clones covers a consensus sequence ( - - - ) of about 45 kb. Regions both heavily over- and underrepresented and even gaps in the consensus sequence represent a situation typically in shotgun projects.

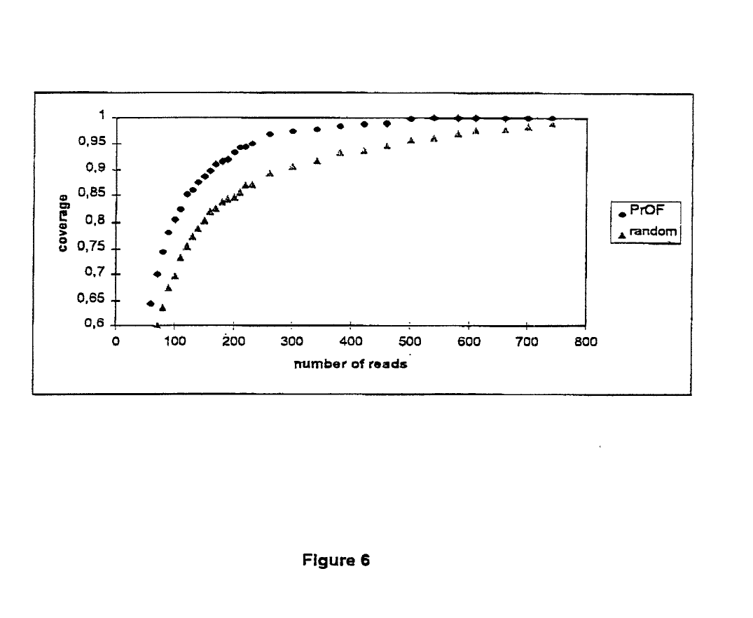

FIG. 6 Quality check of experimental fingerprint data: Comparison between calculated similarity (y-axis) based on hybridization data and real overlap of shotgun clones detected by sequencing (x-axis). The curve represents average values calculated from all clones of this library.

FIG. 6 Graphical representation of the number of reads (x-axis) necessary to achieve a certain percentage of the complete sequence information (y-axis) either used the PrOF approach or random selection.

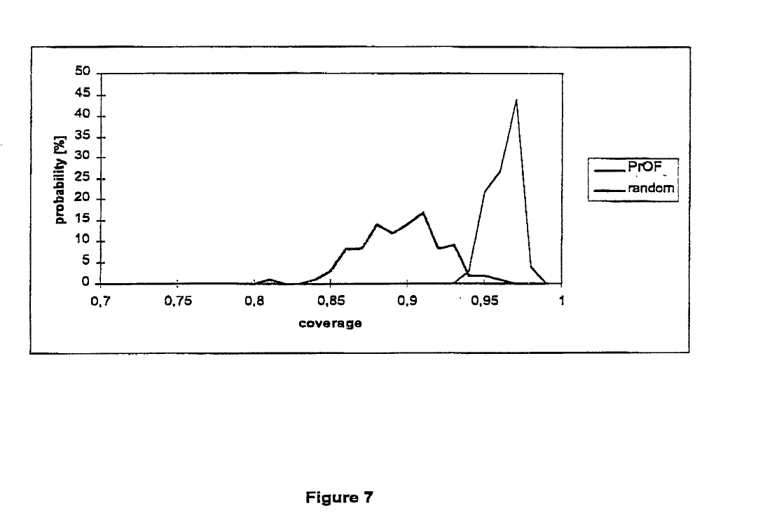

FIG. 7 Graphical representation of the probability (y-axis) to cover a certain percentage of the consensus sequence (x-axis) with a fixed number of 300 reads using either the PrOF approach or random selection.

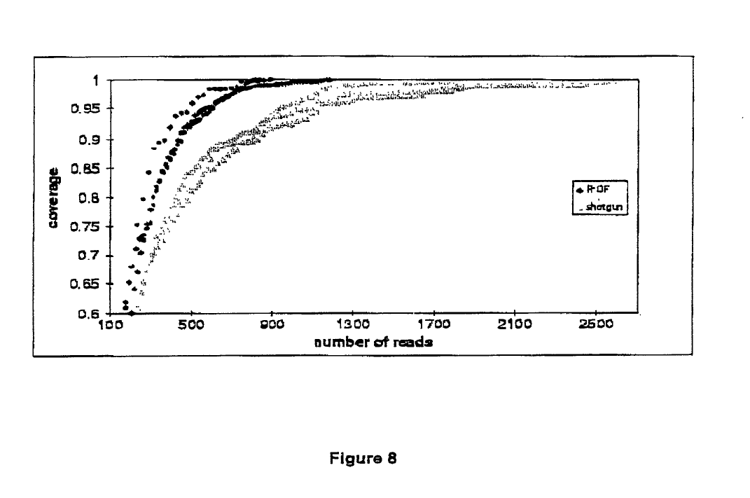

FIG. 8 Graphical representation of the number of reads (x-axis) in the same order as they were actually selected and sequenced. The percentage of the genomic region covered by the respective number of reads is given on the y-axis.

The Examples Illustrate the Invention.

### EXAMPLE 1

### Generation of Shotgun Libraries

PAC DNA is prepared as described in (31), purified by alkaline lysis and caesium chloride banding, and then sheared by sonication, The resulting DNA fragments are end-repaired, size-selected, ligated into SmaI digested and dephosphorylated pUC18 vector and transferred by electroporation into E. coli (strain KK2186). The bacterial suspension is plated out on 22 cm×22 cm LB-Agar plates containing ampicillin, X-gal and IPTG. Plates are afterwards incubated for 12 hours at 37° C. and stored for better development of the blue color for 24 hours at 4° C.

Well separated, white colonies are picked by a robotic picking system (Genetix or Linear Drives) originally developed as described in (32, 33). For each 100 kb to be sequenced ca. 2600 colonies are picked. About 3000 colonies per hour are transferred into 384-well plates containing 2YT media, 100 μg/ml ampicillin and 1 ml/10 ml HMFM freezing solution. After incubation at 37° C. overnight, plates are replicated, incubated again for 18 to 20 h at 37° C. and stored at −80° C.

### EXAMPLE 2

### Generation of PCR Products

The hybridization of short oligonucleotides requires highly purified target DNA. This is generated by an automated Polymerase Chain Reaction (PCR) approach on several shotgun libraries in parallel, PCR amplifications are carried out in 384-well microtiter plates (Genetix), in a PCR-thermocycler allowing up to 51,840 PCR amplifications per run. Using disposable plastic 384-pin inoculation devices (Genetix), a small amount of the bacterial suspension (about 0.2 μl) is added to a 40 μl reaction volume containing 50 mM KCl, 10 mM Tris/HCl, pH 8.5. 1.5 mM MgCl₂, 200 μM dNTPs. 10 pmol of each PCR primer (M13 forward (32mer: [gctattacgccagctggcgaaagggggatgtg]) and M13 reverse (32mer: ccccaggctttacactttatgcttccggctcg) and 0.5 units Thermus aquaticus (Taq) DNA polymerase. After inoculation, the micrometer plates are sealed using a 0.45 mm thick plastic foil with a heat sealer designed for this purpose (Genetix). PCR is performed for 30 cycles consisting of 10 sec at 94° C., 1 sec at 73° C. and 3:30 min at 72° C.

### EXAMPLE 3

### Spotting of PCR Products

High density filter arrays of PCR products from shotgun clones are generated robotically as described previously (Meier-Ewert S. et al., Nucleic Acids Res. 26(9) (1998), 2216). Each 22 cm×22 cm nylon membrane carries 27,648 different clone spots as duplicates. The spots are arranged in 2304 blocks each with 24 spots and with a spot of genomic salmon sperm DNA with the concentration of 600 mg/μl in the center of the blocks. These spots yield signals in every oligo-hybridization experiment and are necessary as guide spots for the automated image analysis. To obtain a quality assessment of the hybridization data, PCR products from previously sequenced shotgun clones are spotted on each filter. The hybridization signals of these clones can thus be directly compared to those predicted from the DNA sequences.

After spotting the nylon filters were stored in 22.5×22.5 cm plexiglasboxes at 4° C. The permanent immobilization of DNA comprises the following steps:

1. Laying the nylon filter on a 0.4 M NaOH solution for 2 min (not submerging);

2. Submerging the nylon filter in 5× SSC solution for 2 min;

3. Air-drying the filter after laying on 3MM-Whatman-paper for 1 h at room temperature;

4. Incubating the filter for 30 min at 80° C.; and

5. Crosslinking the filter with UV radiation (UV-Stratalinker 2400, Strategene). 20 filter copies are prepared for parallel hybridization experiments.

### EXAMPLE 4

### Oligonucleotide Hybridization

Using a computer program developed in-house (see below) a set of 100 8mer oligonucleotides, best suited for characterization of genomic DNA, were selected out of a set of more than 250 oligonucleotides used in our laboratory for characterization of cDNA libraries.

The selection algorithm of that program is based on the concept of entropy of information theory. For a given set of n oligonucleotides there are 2ⁿ possibilities to hybridize or not to a clone. Each of these possibilities has a probability pₗ. The entropy of the set of oligonucleotides is then defined by Σₗⁿ pₗlnpᵢ. The probabilities are estimated by the relative frequencies of hybridization of the oligonucleotides in a set of clones created by cutting several Mb of genomic sequences from commonly available databases into pieces of typically sized shotgun clones, e.g., 1-2 kb. The program tries to select the set of oligonucleotides which maximizes the entropy.

Since 10mers hybridize more reliably than 8mers each probe in reality comprises a pool of all 16 10mers sharing the same 8mer core sequence with “N”s at the 3′ and 5′ ends (NXXXXXXXXN). Each of the oligonucleotides was hybridized in a separate experiment. Thus, for characterizing the clones spotted on the filter, 100 hybridizing patterns were generated with 100 oligonucleotides.

The oligonucleotides are labeled at the 5′ end by a kinase reaction using [γ-³³P]ATP (Amersham International) and T4 polynucleotide kinase (New England Biolabs). 30 pmol of the oligonucleotide was labeled in a reaction volume of 30 μl. The reaction mixture contained 10 μl H₂O, 3 μl 10×T4-kinase-buffer (New England Biolabs), 2 μl T4-kinase [10U/μl] (New England Blolabs) and 5 μl [³³P-8]ATP [10 μCi/μl] (Amersham International) for the labeling of 10 μl of the oligonucleotide. The reaction mixture was incubated at 37° C. for 30 min to 1 h. If not used immediately, the mixture was stored at −20° C. for a max. 10 days. Each probe is used in a separate hybridization experiment. Using 20 filter copies 20 hybridizations are carried out in parallel. The filters are prehybridized with a buffer containing 600 mM NaCl, 60 mM sodium citrate, 7.2% Na-Sarkosyl (SSarc-buffer) for 10 min. The hybridizations are performed overnight at 4° C. in hybridization bottles containing 12 ml SSarc-buffer with a probe concentration of 2.5 nM. Afterwards 10 filters are washed at a time in 1 l of the same buffer for 20 min at 4° C. To evaluate the total amount of DNA which has been spotted for each clone on the filter, on additional hybridization is carried out with a 11mer oligonucleotide matching plasmid vector sequence common to all PCR products.

To remove the fixed radioactive oligonucleotides on the filter 20 filters were incubated twice in 1 l 0.1× SSarc at 65° C. for 20 min.

The intensities of the hybridization signals are measured by a phosphor storage autoradiography (Molecular Dynamics, Sunnyvale, Calif.). The system is at least ten times more sensitive and faster than conventional film-based autoradiography and allows linear measurement of the hybridization signal over a larger range (Johnston R. F. et al., Electrophoresis 11 (1990), 355). The phosphor imager scans with 16 bit gray scale resolution and with a resolution of 88 or 176 μm per pixel. The result is subsampled to an 8-bit 1024×1024 image. It requires about 5 min to scan a 22×22 cm hybridization image, allowing the subsequent scanning of many filter images a day.

### EXAMPLE 5

### Re-arraying and Sequencing of Clones

Clones selected for sequencing are collected with a re-arraying robot and sequenced. The robot takes the clones out of the 384-well microtiter plates and puts them into specified positions in 96-well microtiter plates, which are forwarded to the sequencing unit. The robot routinely re-arrays more than 600 clones per hour without cross contamination and with a yield of more than 97%, i.e. less than 3% of the bacterial clones fail to grow in the daughter plates (Radelof, Nucl. Acids Res. 26 (1998), 5358-5364).

The sequencing reactions are carried out using dye primer technique on an ABI catalyst robot using 1 μl of the PCR product and 3 μl of the ThermoSequenase mix (Perkin Elmer) for each of the four A; C; G; T reactions. Energy transfer primer (0.1 pmol for A, C and 0.2 pmol for G, T reactions respectively) M13(-40) or M13(-28) were added to the ThermoSequenase mix before starting the sequencing run. Samples are pooled and precipitated according to ABI's instructions and analyzed on ABI 377XL DNA sequencers. Data were processed using ABI's sequence analysis software version 3.0 and 3.1, but with the Perkin Elmer manual lane tracking kit according to the manufacturer's instructions.

### EXAMPLE 6

### Image Analysis

Hybridization images obtained from the phosphor imager are transferred to a DEC alpha UNIX workstation. An image analysis program determines raw hybridization intensities for each clone and probe and substracts the average background from the signals A normalization routine compensates for 1. different overall hybridization Intensities (maxima and minima) from different probes and 2. different masses of different clones. The final output is a hybridization matrix containing normalized Intensities for all clones and probes. An example is given in table 1. Each row of this matrix represents the oligofingerprint of one clone. Programs for hybridization data analysis on high density matrices were written in our laboratory.

A large number of clones are hybridized in parallel with radioactive labeled probes.

The image analysis program assigns each clone on the filter an intensity value that should be proportional to the bounded radioactivity of the probe.

The image processing performs the following tasks:

1. Subtract the local background

2. Find the spot positions

3. Cross talking algorithm to correct overshining effects

1. Subtract the Local Background

The next step is the subtraction of the background intensity. This intensity is not determined for the filter as a whole but locally for each pixel. The intensity which is higher than 15% of the intensities of the square is assumed to be the local background intensity. Each pixel can be considered as the center of a square with the size of 40×40 pixel. These squares overlap with some of the initially constructed. The background intensity of these squares is then multiplied with the relative overlap and subtracted from the pixel intensity.

2. Spot Finding

In order to find the spot positions the first task of the image analysis program is to find the blocks by determining the guide spot positions. Currently this task Is not performed in a fully automatic procedure. The corners of the filter are found visually. Using this information the guide spot positions are found by a simulated annealing algorithm. Two factors are considered in the definition of the quality function: The deviation of the distances of the guide spot position from its specified value and the intensity value of the pixel at the assumed position of the guide dot. The deviation of the distances should be very small whereas the intensity at the guide spot positions should be high.

The procedure is initially performed for the whole filter. Then the results will be adjusted for each field.

Once the guide dots are found, the spot position will be determined by the specified grid.

3. Cross Talking

Finally a cross talking procedure is performed to compensate the overshining of a spot by its neighboring spots. This effect is calculated by the comparison of the real spot shape with the theoretical spot shape.

TABLE 1

| | oligo 1 | oligo 2 | . . . | | |
|---|---|---|---|---|---|
| clone 1 | 0.00000 | 2.873524 | 0.00000 | 3.211587 | 0.00000 |
| clone 2 | 0.00000 | 0.00000 | 0.00000 | 0.00000 | 0.00000 |
| . . . | 0.00000 | 0.00000 | 2.028370 | 0.00000 | 0.00000 |
| | 1.183216 | 0.00000 | 0.00000 | 0.00000 | 0.00000 |
| | 2.535463 | 0.00000 | 0.00000 | 0.00000 | 0.00000 |
| | 0.00000 | 0.00000 | 0.00000 | 0.00000 | 0.00000 |
| | 0.00000 | 0.00000 | 0.00000 | 0.00000 | 0.00000 |
| | 0.00000 | 0.00000 | 0.00000 | 2.525463 | 0.00000 |
| | 0.00000 | 1.690309 | 0.00000 | 0.00000 | 0.00000 |
| | 0.00000 | 0.00000 | 0.00000 | 3.380617 | 0.676124 |
| | 0.00000 | 0.00000 | 0.00000 | 0.00000 | 0.00000 |
| | 1.183216 | 0.00000 | 0.00000 | 0.00000 | 0.00000 |
| | 0.00000 | 0.00000 | 0.00000 | 3.192181 | 0.00000 |
| | 0.00000 | 0.00000 | 0.00000 | 3.380617 | 0.00000 |
| | 2.028370 | 0.00000 | 0.00000 | 2.028370 | 0.00000 |
| | 0.00000 | 0.169031 | 0.00000 | 0.00000 | 3.380617 |
| | 0.00000 | 0.00000 | 0.00000 | 0.00000 | 0.00000 |
| | 0.00000 | 3.042555 | 0.00000 | 0.00000 | 0.00000 |
| | 0.00000 | 0.169031 | 0.00000 | 0.00000 | 1.859339 |
| | 0.00000 | 0.00000 | 0.00000 | 3.038851 | 0.00000 |

Excerpt of a typical fingerprint matrix containing the hybridization intensities of each clone and probe (oligonucleotide). Data are filtered with respect to background noise and are normalized.

### EXAMPLE 7

### Preselection

The aim of the present invention, namely of the preselection is to avoid unnecessarily high sequencing redundancy. Therefore, we search for shotgun clones representing a minimum tiling path along the pool of more or less randomly distributed shotgun clones representing the entire sequence of the original genomic clone. The clones required have minimal sequence overlaps, indicated by maximally dissimilar hybridization patterns. The results of the preselection procedure is a list of clone names which indicates the position of the corresponding PCR-amplifications in a 384-well microtiter plate (Genetix).

Single clones can be identified by their fingerprint vector F_{N}, which contains the hybridization intensity for oligos J=1, . . . , K on clone N. A simple measure for the similarity of two vectors is their scalar product:

$${S}_{NM} = {\vec{F}}_{N} \cdot {\vec{F}}_{M} = {\sum }_{j = 1}^{K} {F}_{NJ} \cdot {F}_{MI} ε {801}^{ZCode} bA {802}^{ZCode} B {803}^{ZCode}$$

Two vectors (clones) can be regarded as maximally dissimilar, if S_{NM}=0, i.e. they have no oligonucleotide match in common, and as maximally similar, if S_{NM}=1 (for normalized fingerprint vectors).

Once the scalar product for each clone pair is calculated the construction of a low redundancy set can be done using the following series of steps:

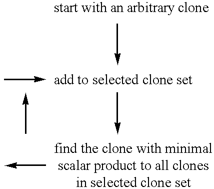

The selection of a typically sized set from a shotgun library containing 2600 clones for a 100 kb PAC Is completed in a few minutes on a standard UNIX workstation.

### EXAMPLE 8

### Simulation Experiments

Different computer simulations were carried out in order to compare the efficiency of the preselection under various conditions with the standard shotgun approach. The influence of the shotgun clone insert size, the insert size distribution and the repeat content of the genomic region in question have been investigated. For this purpose arbitrarily chosen human genomic sequences of 100 kb length were extracted from a publicly available database (http:/www-eri.uchsc.edu/chr21). and randomly cut into pieces of typical shotgun clone sizes. But some arbitrarily chosen areas were set to over- or underrepresented regions based on typical assemblies of sequenced shotgun libraries. Each virtual shotgun library consisted of 2000 clones. Theoretical oligofingerprints were generated using the same set of 8mer oligonucleotides applied in the real experiments. Hybridization “intensities” were set to 1 in cases where the oligonucleotide sequence matched the clone sequence, and to 0 otherwise. The real situation is more complicated since 7 (1 mismatch) and even multiple 6 (2 mismatches) matches yield strong signals and float numbers of signal intensities are used.

In all simulations shotgun clones were selected using the selection algorithm given in Example 7. The same numbers of clones were taken by a random process simulating shotgun sequencing. All clones selected were “virtually” sequenced from both sides with a read length of 600 bases. After assembly the consensus sequence was measured and compared (FIGS. 1 to 3). Each point in the curves represent an average value of 50 statistically independent selected clone sets.

In the first simulation experiment (FIG. 1) the influence of the amount of repetitive sequences of the genomic region (cosmid. PAC, etc.) to be sequenced was examined. For this a 100 kb database sequence with an amount of repetitive sequences of 52% (ALU, LINE, MER, etc.) was used in comparison to an artificial repeat-free sequence of the same length. This sequence was constructed by combining several repeat-masked database sequences. In both cases shotgun clones of fixed size (1.5 kb) were used.

ALU-elements are one of the most repetitive sequences in human genomic DNA with a length of 300400 bp (Jurka, Journal of Molecular Evolution 32 (1991), 105-121). Typical shotgun-clones are 1-2kb in length. Thus, there is always enough sequence information provided to distinct clones derived from different regions containing ALU-elements by their oligofingerprints, if enough oligonucleotides are used.

LINE-elements belong to a further family of repetitive sequences and are found up to 7 kb in length (Jurka, Journal of Molecular Evolution 29 (1989), 496-503). However, since LINE-elements occur in very different ways within the human genome, clones derived from different LINE-regions can be distinguished from each other according to their oligofingerprints.

However, a large amount of repetitive sequences within a genomic region will on average reduce the effectiveness of preselection.

Problems can arise when duplicated regions with several kb in length are to be sequenced. In this case there is no possibility to determine the position of a shot-gun clone within the genomic sequence according to its oligofingerprints. Nevertheless, it is unlikely to have these problems when working with cosmid- or PAC-Clones. Accordingly, the invention will work suboptimally only in rare cases.

In the second experiment (FIG. 2) the same sequence containing 52% repetitive sequences as above, was “shotgunned” into clones of either fixed or Gaussian distributed insert length.

In the third experiment (FIG. 3) again the sequence containing 52% repetitive sequences was used to consider the impact of the shotgun clone insert size using shotgun clones of different but fixed sizes. The differences in efficiency of the PrOF method in all test cases are very small, indicating that the influence of these parameters is weak, and demonstrating the robustness of the fingerprint approach. In the region around 97% coverage of the entire genomic sequence where usually the “gap closure” starts, the PrOF approach required in all cases considered, much less than half the number of sequence reads compared to random selection.

### EXAMPLE 9

### Pilot Experiment

In order to test the efficiency of the PrOF strategy for handling experimental data, an already sequenced cosmid shotgun library containing about 40% repetitive sequences (ALU, MER, etc.) was used. FIG. 4 shows the assembly of 426 clones covering a consensus sequence of about 45 kb. The assembly does not contain the finishing data produced by primer walking. Large fluctuations in coverage clearly reflect a situation typical in shotgun projects, with regions both heavily over- and underrepresented and even with gaps in the consensus sequence due to statistical and biological effects.

In the conventional shotgun approach a large number of randomly chosen clones are sequenced in order to increase the probability of obtaining sequences in underrepresented regions. However, this strategy also increases the mean coverage to unnecessarily high values, In the present example, the average coverage is 11 fold, with maximal local coverage around 30 fold. The generation of so many sequence reads and the additional gap closure makes the process much more expensive than it need be, blocks sequencing capacity and wastes time.

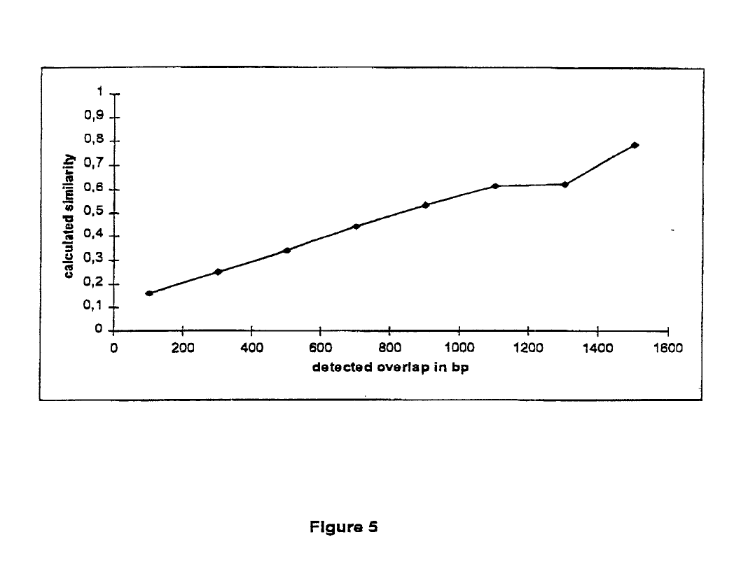

All shotgun clones of this library were PCR amplified, spotted on filters and oligofingerprints were created as described in the previous Examples. As a quality check of the experimental fingerprint data the calculated similarity of the clones were compared using hybridization data with the real clone overlap detected by sequencing. The observed relationship is nearly linear as shown in FIG. 5.

For a direct comparison of the PROF approach with the random approach used in the standard shotgun procedure, certain numbers of clones were selected out of the same clone pool either based on oligofingerprints or randomly (FIG. 6). Again as in the simulations, in the region around 97% coverage, the PROF method is about two-fold more effective than the random selection (table 2).

TABLE 2

| COVERAGE | RANDOM | PrOF | |
|---|---|---|---|
| [%] | [READS] | [READS] | RANDOM/PrOF |
| 90 | 286 | 164 | 1,74 |
| 96 | 542 | 248 | 2.18 |
| 97 | 588 | 276 | 2.13 |
| 98 | 685 | 364 | 1,88 |

Number of reads required to gain a certain percentage of the genomic sequence covered are given for the PrOF approach and the random selection. Ratios of reads required are also shown.

Each point of the curves in FIG. 6 represents an average of 50 statistically independently selected clone sets. In each single experiment a different result is achieved. In one experiment possibly 300 reads are needed to achieve 97% coverage, while in another 270 or 330 could be necessary to cover the same consensus sequence. The range of variation at a fixed set size is given in FIG. 7 for both methods. The PrOF method clearly shows a much more narrow variation. The certainty of getting a specific coverage in a single experiment is much greater in comparison to the random approach.

### EXAMPLE 10

### Application in large-scale Sequencing

The preselection strategy was applied to a large-scale sequencing project spanning a 1.5 to 2 Mb region of the 17p11.2 region of the human genome. In the first experiment we are using 5 shotgun libraries derived from PAC's between 70 and 130 kb in size, 535 kb in total. All amplified clones are spotted on one filter (20 filter copies). In addition, clones from 5 already sequenced cosmid derived libraries are spotted on the same filter as controls. After the hybridization of 100 oligonucleotides (20 in each step in parallel, using 20 filter copies) and the computational analysis of 82 hybridization images (18 low quality images rejected) the selected clones were robotically re-arrayed and sequenced from both sides.

In 4 out of 5 preselectlon projects almost the same results as in the simulations and the pilot experiment were obtained. FIG. 8 depicts the results from 3 of these projects in direct comparison to 3 typical shotgun projects (also PAC derived) carried out simultaneously. In order to normalize the results to a common scale, the number of all sequence reads is divided by the respective PAC size and multiplied by 100 kb. Again, as it is shown in table 3 in the projects where the PrOF strategy was used only half the number of sequences reads as necessary, compared to the standard shotgun projects, to get the same consensus sequence length.

TABLE 3

| COVERAGE | SHOTGUN | PrOF | SHOTGUN/ |
|---|---|---|---|
| [%] | [READS] | [READS] | PrOF |
| 90 | 771 | 416 | 1.85 |
| 96 | 1132 | 581 | 1.95 |
| 97 | 1263 | 614 | 2.05 |
| 98 | 1523 | 677 | 2,25 |
| 99.5 | 2003 | 851 | 2.35 |

Number of reads required to gain a certain percentage of the genomic region covered are given as average values for the projects depicted in FIG. 8. Ratio of reads required to cover the same consensus sequence length is also shown.

### EXAMPLE 11

### GAP Closure with Specific Clone Selection

With sequencing of preselected shotgun-clones no sequence was obtained covering the whole genomic region. The phase of gap closure in traditional shotgun sequencing cannot be eliminated yet. However, this method can simplify and accelerate the phase of gap closure. Due to the oligofingerprints, clones can be selected that enlarge sequence contigs or cover gaps. To prove the method gaps were introduce into existing sequence contigs by removal of clones out of the assembly in computer experiments. The removed clones were given back into the pool of clones of the original shotgun library, Then the removed clones were “fished” by the oligofingerprints of those clones that remained at the end of the contig. To improve the possibility of selecting clones closing the gap those clones were not included into the search whose fingerprints are closest to the target clone overlapping the contig. The gaps could be closed by the selected clones.

1. A method of the preselection of shotgun clones of the genome on a portion of a genome of an organism comprising:
(a) providing a shotgun library of said genome or said portion of the genome;
(b) amplifying said library by an amplification method;
(c) transferring clones of said library onto a carrier;
(d) optionally, generating one or more replicas of said carrier;
(e) allowing binding a set of labeled or unlabeled probes
(i) sequentially to said clones on said carrier or clones on replica(s) of said carrier(s); or/and
(ii) to clones on said carrier and to clones on replicas of said carrier or to clones on replicas of said carrier;
(f) detecting clones that bind to one or more of said probes,
(g) optionally, evaluating the signal intensity of said binding;
(h) selecting a number of clones that were detected in step
(f) or evaluated in step (g), wherein
(i) each of said clones binds with at least one different probe of said set of probes; or
(ii) clones that bind to the same probes from said set of probes generate different signal intensities in the binding signal with at least one probe from said set of probes; and
wherein the sum of the basepairs of the inserts of said shotgun clones at least equals the number of basepairs of the genome or investigated part of the genome of said organism.

2. The method of claim 1, wherein said DNA amplification to step (b) is effected by polymerase chain reaction.

3. The method of claim 1 or 2, wherein said organism is a human, mouse, zebrafish, drosophila, amphioxus, yeast, arabidopsis, meningococcus or plant or fungi or microorganism.

4. The method of claim 1, wherein said shotgun library is provided in a storage compartment.

5. The method of claim 4, wherein said storage compartment is a microtiter plate.

6. The method of claim 1, wherein said probe is an oligonucleotide which comprises between 2 and 50 nucleotides.

7. The method of claim 6, wherein said probe is an oligonucleotide which comprises between 6 and 10 nucleotides.

8. The method of claim 1, wherein said carrier is a planar carrier.

9. The method of claim 8, wherein said planar carrier is a membrane, or filter, or chip, or beads, or glass, or silicon, or metal, or plastic or ceramics, or specifically treated or coated versions of the aforementioned.

10. The method of claim 9, wherein said planar carrier is a filter and said filter is preferably a nylon filter or nylon membrane, a PVDF-membrane or a glass (specifically coated).

11. The method of claim 1, wherein said transfer in step (c) is made or assisted by automation, a spotting robot, pipetting or micropipetting device.

12. The method of claim 1, wherein said transfer is in a regular grid.

13. The method of claim 12, wherein said regular grid has densities of 1 to 1,000,000 spots.

14. The method of claim 13, wherein said regular grid has densities of 1 to 10,000 spots of PCR products (or otherwise generated nucleic acid fragments) of shotgun clones per square centimeter.

15. The method of claim 1, wherein said probes are labeled with a radioactive, a chemiluminescent, a fluorescent, a phosphorescent marker or a mass label.

16. The method of claim 1, wherein said detection is effected by digital image storage, analysis, processing or visual imaging or mass spectrometry.

17. The method of claim 1, wherein said set of oligonucleotides comprises between 10 and 10,000 different probes.

18. The method of claim 1, wherein in step (d) between 1 and 10,000 replicas are generated.

19. The method of claim 1, wherein in step (d) between 2 and 10,000 different replicas are generated.

20. The method of claim 1, wherein the sum of basepairs of said inserts amounts to 1 to 30 times the number of basepairs in the genome or said portion of said genome of said organism.

21. The method of claim 20, wherein the sum basepairs of said inserts amounts to 2 to 4 times the number of basepairs in the genome or said portion of said genome of said organism.

22. The method of claim 1, wherein said probe is PNA oligonucleotides or pools of DNA and/or PNA oligonucleotides, antibodies, fragments or derivatives thereof.

23. The method of claim 1 further comprising:
(i) sequencing clones selected after hybridizing to said oligonucleotides.

24. The method of claim 1, wherein said probe, preferably said oligonucleotide recognizes a contiguous or non-contiguous region of between 2 and 30 nucleotides.

25. The method of claim 1, wherein each clone binds to a different subset of probes indicating minimal overlap to previously selected clones based on appropriate statistical criteria to produce a minimal overlapping clone set.

26. A method of the production of a pharmaceutical composition comprising formulating an open-reading frame comprised in a clone selected after hybridizing to one of said oligonucleotides or an expression product thereof in a pharmaceutically acceptable form.
